(19) United States
(12) Patent Application Publication
(10) Pub. No.: US 2003/0190344 A1
(43) Pub. Date: Oct. 9, 2003

(54) Animal feed or feed additive as performance enhancer or appetite enhancer for live stock

(76) Inventor: Hermann Roth, Eltville (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/380,447

(22) PCT Filed: Sep. 14, 2001
(86) PCT No.: PCT/IB01/02487

(30) Foreign Application Priority Data
Sep. 15, 2000 (AT) A 1579/2000

(51) Int. Cl. A61K 31/4745; A23K 1/165; A23K 1/17

(52) U.S. Cl. 424/442; 514/285

(57) ABSTRACT

The invention relates to an animal feed which contains conventional animal feed substances like grain or grain products, maize, protein and aromatic amino acids, vitamins, mineral additives like salts, phosphates, lime, enzymes and the like, or an animal feed additive, whereby to reduce the enzymatic decomposition of the essential aromatic amino acids it contains one or more benzophenanthridine alkaloids or their derivatives or synthetic analogs in an effective amount. Furthermore the invention relates to the use of benzophenanthridine alkaloids, their derivatives or synthetic analogs and a medicament.

The invention relates to an animal feed which contains conventional animal feed substances, like grains or grain products, maize, protein and aromatic amino acids, vitamins, mineral additives like salt, phosphates or lime, enzymes and the like. The invention further relates to animal feed additives, the use of benzophenanthridine alkaloids and to a medicament.

Aromatic amino acids, like for example tryptophane and alanine, are essential nutrients which the body itself cannot produce, except for ruminants. They are a substantial component of the recipes for nutrients and animal feeds. In animal feeds they serve to ensure, as is also the case for other amino acids, the requisite balance between the individual essential and nonessential amino acids in a predetermined proportion to the content of other essential amino acids. Their use contributes to the cost.

In mammalian waste there is a wide range of microorganisms which with the aid of their enzymes, serve to decompose these amino acids, for example the aromatic aminoacid-decarboxylase. As a result, the starting balance which is intended in the preparation of the feed between the individual amino acids is disrupted. The quality of the meat of the animals is thereby reduced.

Indole and skatole arise as intermediate products of the enzymatic decomposition of aromatic amino acids and bring about undesired characteristics and effects. Indole and skatole are toxic substances of tryptophane decomposition. They provide a strain on the liver among other things since the body must detoxify these substances to avoid illness. Indole and skatole give rise to chronic subclinical inflammation of the mucosa of mammalian animals and humans. Indole and skatole produced by microorganisms of the intestinal mucosae facilitate the invasion of pathogenic parasites (intestinal worms, Clostridiae, Salmonellae, Coccidiae) in the upper cells of the intestinal mucosa or avoid the defense of the body against such pathogens.

Furthermore, a residue of indole and skatole in the nutrients can also detrimentally affect the health of consumers or their well being. An unsatisfactory relationship between aromatic amino acids (with respect to quantities which may be too low) and the other essential amino acids give rise to imbalance which has the effect of reduced protein content and a reduced lactalbumin.

Nutrient quality also depends on the protein content. In the case of a deficiency of aromatic amino acids and reduced protein content (lactalbumin, lean meat), there is a reduction in quality and hence an impact on the price obtainable.

The aromatic aminoacid-decarboxylase has been found to be a possible source of illness in conjunction with Parkinsonism and can lead directly to exacerbation thereof. A number of micro-organisms which live in the intestines and the intestinal mucosae of animals produce enzymes which decompose proteins and amino acids. The situation is critical when essential nutrients encounter these decomposition processes and especially when toxic decomposition products result which affect the host or give rose to residues in nutrient agents. The cost of animal feed optimization is thus increased as hitherto unavoidable losses of aromatic amino acids require the consideration of increased additives to feeds. As a consequence of the presence of unaffected activity of the aromatic aminoacid-decarboxylase, the cost of feeding and maintaining the health of crop and hobby animals is increased.

The animal feed according to the invention is characterized at a first level in that it contains one or more benzophenanthridine alkaloids or their derivatives or synthetic analogs in an effective quantity for reducing the enzymatic decomposition of the essential aromatic amino acids.

Chelerythrine and sanguinarine as well as other analogous benzophenanthridine alkaloids have been found to be potent inhibitors of the aromatic aminoacid-decarboxylase. It has been found that sanguinarine and chelerythrine can block the activity of the aromatic aminoacid-decarboxylase to 99% or 92%. This blockade of the aromatic aminoacid-decarboxylase is irreversible and thus is of special interest since it is maintained as long as the enzyme molecules are in the effective range of the benzophenanthridine alkaloids. Sanguinarine and chelerythrine, representative of the benzophenanthridine alkaloids, have an aromatic structure which is similar to that of the aromatic amino acids. The enzyme “aromatic aminoacid-decarboxylase” confuses the ultimate structure of the benzophenanthridine alkaloid with the ring structure of the aromatic amino acid and fits it rather than the amino acids at its active center. The benzophenanthridine alkaloid, however, does not have a splitable carbon skeleton like those of the amino acids and thus does not yield any end product of the enzymatic reaction so that these do not terminate. The active centers of the aromatic aminoacid-decarboxylase are irreversibly blocked. The invention shows that it is possible to block irreversibly the activity and the metabolic product formation of the aromatic aminoacid-decarboxylase by the addition of a structure similar to the aromatic structure of the aromatic amino acid and to use this for the animal nutrition and advantageously as a medicament.

The invention gives rise to a reduced decomposition and loss of essential aromatic amino acids and thus to a better supply of the animal with these nutrients. Experiments show that in this manner the lean meat contents and the content of lactalbumin is clearly and to a significant part improved. Further tests have shown that the result is an improved balance within the amino acid pattern of the protein balance in pigs in the final feeding up to about 10%.

The invention gives rise to an improved animal health. Tests show that the use of the benzophenanthridine alkaloids result in a clearly reduced inflammation frequency in the intestinal tracts of animals.

The animal feed according to the invention can contain all conventional feed substances as are usually used in animal husbandry. In this category fall the starch-containing components like grains and grain products and maize. The protein which also can be contained therein can be of natural origin or supplied as preparations. Such preparations can especially provide the essential amino acid. In addition, mineral additives like trace element phosphates, salts and lime can be added. Further additives like enzymes, vitamins or medicaments can also be added.

The aromatic amino acids contained in the animal feed can derive in part either from natural sources or artificial additives. The most important aromatic amino acids are, for example, tryptophane and phenyalanine.

The benzophenanthridine alkaloids in nature come for example from the rhizomes of the Canadian blood-wort Sanguinaria canadensis or from Chelidonium majus or Macleaya cordata. These plants are part of the class papaveraceae. The rhizomes or the leaf material or the entire plant material can be collected and dried. Based upon the dry substances the amount of benzo-phenanthridine alkaloids is usually about 4% by weight. To level out fluctuations in harvesting it can be advantageous to standardize the content of the main alkaloid sanguinaria to a value of, for example, 1.5% by the addition of fillers. Since approximately one-half of the natural alkaloid content is sanguinarin, the total alkaloid content is then about 3% by weight.

Other alkaloids of these groups are chelerythrine, chelirubin, sanguirubin, chelilutin and sanguilutin.

Within the scope of the invention it can also be advantageous to dispense extracts of the plant material or the isolated alkaloids and their derivatives or their synthetic analogs.

The alkaloid quantity contained in the animal feed has its lower limit only determined by the effectiveness. The total alkaloid quantity per ton of the animal feed is preferably greater than 1 mg. Based upon the standardized dried papaveraceae root material, this corresponds to about 0.067 g per ton of animal feed.

In the papaveraceae tested more exact dispensing of the animal feed, amounts between 5 g and 150 g of dried papaveraceae per ton of animal feed, standardized to 1.5% of the sanguinaria alkaloid, corresponding to about 3% total alkaloids were used.

The invention embraces also animal food additives like for example premixes or mineral feeds or completion feeds for the preparation of animal feeds whereby the additives apart from the usual animal feed additive materials contains benzophenanthridine alkaloids or their derivatives and analog or plant parts of the papaveraceae containing these alkaloids.

The invention thus encompasses both the feed supplied to the animal as well as the preproducts which the user can utilize to produce the finished animal feed.

The invention also comprises the use of benzophenan-thridine alkaloids or their derivatives and analogs or plant parts of the papaveraceae containing them for limiting or suppressing the enzymatic decomposition of aromatic amino acids, especially in the intestinal tracts of animals, especially in combination with a previously described animal feed or animal feed additive.

When the animal feed contains sufficient aromatic amino acids, the addition of the alkaloids which result in enrichment of the expensive aromatic amino acids therein can be reduced. To the extent that aromatic amino acids must be added, e.g. tryptophane and phenyalanine, the amounts which are supplied can be reduced by the addition of the alkaloid to save cost. As a basis for evaluation, based upon present day market conditions, a saving of about 10 Euro in the animal feed price can be obtained when the alkaloid preparation of the present invention at a value of 1 Euro is added to the animal feed.

The benzophenanthridine alkaloids can, according to the invention, also be added advantageously as a medicament against intestinal inflammation and especially those which are caused by the decomposition of the amino acid metabolism like indole or skatole. The medicament is characterized by a content of an effective amount of the benzophenanthridine alkaloids or their derivatives or synthetic analog or plant parts of the papaveraceae containing them.

The use of the medicament is effected preferably simultaneously with the animal feed or as an additive to the drinking water. The effective amount of the alkaloids lie in the same ranges as have been given.

Below several examples are described.

### 1. EXAMPLE OF A CONVENTIONAL ANIMAL FEED RECIPE FOR CHICKENS AND PIGS

| | (In weight percent) |
|---|---|
| Wheat: | 42% |
| Maize: | 20% |
| Sunflowers | 3.6% |
| Soy scrap I | 4% |
| Soy scrap II | 16.6% |
| Rape scrap | 4% |
| Fodder oil | 6.5% |
| Methionine | 0.26% |
| Lysine | 0.48% |
| Mono Calcium Phosphate | 0.85% |
| Lime | 0.5% |
| Salt | 0.025% |
| Premix | 1.0% |
| Enzymes | 0.2% |
| Amino Acids | |
| Protein | 19.2% |
| Lysine | 0.824% |
| Methane | 0.276% |
| Meth + Cystine | 0.568% |
| Threonine | 0.08% |
| Tryptophane | 0.24% |

### 2. EXAMPLE ACCORDING TO THE INVENTION

The animal feed in pigs corresponding to FIG. 1. The given amounts of papaveraceae were added in grams of the dried root material with 1.5% by weight of the sanguinarine alkaloid per ton of the animal feed. The animal species was: Great Yorkshire plus crossing sows. The amounts were per group of 23-24 animals.

| | | Group of Test Animals | | |
|---|---|---|---|---|
| | | a | b | c |
| Papaveraceae g/t | Starter/Prefeed 22 kg-48 kg | 0 | 0 | 15 |
| | Final Feed 48 kg-109 kg | 0 | 30 | 30 |

Results:

Classification and Slaughtered Body Value

(AA Better Than A Better Than B):

| | |
|---|---|
| | Papaveraceae-Dose g/t |
| 0 Control | Prefeed/Final Feed |

| | (0/0) | (0/30) | (15/30) |
|---|---|---|---|
| Lean meat % | 54.5 | 55.1 | 55/0 NS |
| Back bacon mm | 17.8 | 17,7 | 17.6 NS |
| Muscle area | 51.9 | 55.6 | 54.2 (p < 0.05) |
| Class AA + A % | 77% | 96% | 92% |
| Class B | 23% | 4% | 8% |

Thus with the invention the preparation of Class AA+A is significantly increased.

### 3. EXAMPLE

| | | |
|---|---|---|
| Groups: | 0-Control | 30 Papaveraceae/t Animal Feed |

144 piglets at start, 72 per group, distributed
in 8 repetitions per group.
Restrictive feeding.

Prefeed—Final Feed 31-115 kg:

| | 0-Control | 30 Papaveraceae 30 g/t |
|---|---|---|
| Lean meat (FOM) % | 64.9 | 55.2 |
| Muscle area cm² | 51.7 | 52.0 |
| Back bacon cm | 20.6 | 20.1 |

Economics: Basis 1.022 Euro/kg Slaughtered Weight

| | |
|---|---|
| Slaughtered body value Euro | +1.63 |
| Papaveraceae Profit Euro | +2.71 |

Commentary:

In a system with restrictive feeding the papaveraceae increases the slaughtered body weight in that the proportions of lean meat and muscle area increase and at the same time the back bacon value drops. Thus the papaveraceae increases the meat quality and the profitability by about 1.53 Euro per animal.

### 4. EXAMPLE

| | Control | |
|---|---|---|
| | 15 ppm Virginiamycin | Papaveraceae |
| | (Antibiotic) | 50 g/t |
| Parasite spores in 1000/g | 5 0 40 | 1-6 |
| of dung (coccidiae-cocysts) | | |
| Lesion index 0. * to *** | 0 to ** | 0 to * |

The tests showed a clear and significant improvement in combating intestinal inflammation and a clear reduction in the defecation of intestinal parasites.

1. An animal feed which contains conventional animal feed substances like grains or grain products, maize, proteins and aromatic amino acids, vitamins, mineral additives like salts, phosphates, lime, enzymes and the like or an animal feed additive for producing such an animal feed, characterized in that to reduce the enzymatic decomposition of the essential aromatic amino acids, it contains an effective amount of one or more benzophenanthridine alkaloids or their derivatives or synthetic analogs.

2. An animal feed or animal feed additive according to claim 1, characterized in that the benzophenanthridine alkaloids are present in the form of plant material of the papaveraceae or extracts thereof or in the form of the isolated alkaloids or alkaloid mixtures or synthetic alkaloids.

3. The animal feed or animal feed additive according to claim 1 or claim 2, characterized in that it contains essential aromatic amino acids and benzophenanthridine alkaloids.

4. The animal feed or animal feed additive according to one of claims 1 to 3, characterized in that essential aromatic amino acids are added to the animal feed.

5. The animal feed or animal feed additives according to one of the claims 1 to 4, characterized in that the amount of alkaloid per t of the animal feed is greater than 1 mg.

6. The use of benzophenanthridine alkaloids or their derivatives or synthetic analogs or papaveraceae plant parts containing same for blocking or suppressing the enzymatic decomposition of aromatic amino acids, especially in the intestinal tract of animals and especially in combination with an animal feed or animal feed additive according to one of the claims 1 to 5.

7. A medicament against intestinal inflammation, especially such as is produced by the decomposition products of amino acid metabolism like indole or skatole, characterized by a content of an effective amount of benzophenanthridine alkaloids or their derivatives or synthetic analogs or plant parts of papaveraceae containing them.
